(19) United States
(12) Patent Application Publication
(10) Pub. No.: US 2013/0030415 A1
(43) Pub. Date: Jan. 31, 2013

(54) Urinary catheter that prevents bladder infections

(71) Applicant: Raymond Gordon Britt, Roy, NM (US)

(72) Inventor: Raymond Gordon Britt, Roy, NM (US)

(21) Appl. No.: 13/136,220

(22) Filed: Jul. 27, 2011

(51) Int. Cl. A61M 27/00

(52) U.S. Cl. 604/544

(57) ABSTRACT

Natural urination washes out the pathogens in the urethra, preventing bladder infections. Urinary catheters used currently prevent this flushing action allowing bacteria to progress from the exterior and into the bladder. This invention is a urinary catheter that permits washing of the urethral walls to flush out pathogens. A variety of constructs is to accommodate gender differences and other variables.

### BACKGROUND OF THE INVENTION

Many millions of persons lose the ability to urinate, temporarily or permanently. When the ability to urinate is lost, the solution is to insert a catheter into the urethra and into the bladder to permit urination. The Egyptians used a hollow reed, but today, catheters are made of a variety of materials such a rubber or vinyl. A urinary catheter may be used for each urination, or implanted using a bubble of air or liquid to prevent it from falling out. Whatever style of catheter is used, bladder infections inevitably result, typically within a week to ten days. Before antibiotics were available, loss of the ability to urinate usually resulted in a fatal infection within a few years. Today, antibiotics usually work to eliminate the infection. Use of antibiotics to prevent infection tends to result eventually in the production of antibiotic-resistant bacteria. Therefore, while antibiotics are lifesavers, it is a long and expensive battle for the catheter user, a battle sometimes lost. Urinary catheters are the number one cause of infections in hospitals, despite diligent attempts to avoid them.

The Center for Disease Control article entitled “Engineering out the Risk of Infection with Urinary Catheters” is available at http://www.cdc.gov/ncidod/eid/vol7no2/maki.htm and has copious information and references on this topic. Below is the synopsis of that article.

Catheter-associated urinary tract infection (CAUTI) is the most common nosocomial infection. Each year, more than 1 million patients in U.S. acute-care hospitals and extended-care facilities acquire such an infection; the risk with short-term catheterization is 5% per day. CAUTI is the second most common cause of nosocomial bloodstream infection, and studies suggest that patients with CAUTI have an increased institutional death rate, unrelated to the development of urosepsis. Novel urinary catheters impregnated with nitrofurazone or minocycline and rifampin or coated with a silver alloy-hydrogel exhibit antiinfective surface activity that significantly reduces the risk of CAUTI for short-term catheterizations not exceeding 2-3 weeks.

Millions more, such as myself, use urinary catheters at home and consume vast amounts of antibiotics in a constant war against infection.

The CDC article Biofilms and Device-Associated Infections is available at http://www.cdc.gov/ncidod/eid/vol7no2/donlan.htm. Below is the synopsis Microorganisms commonly attach to living and nonliving surfaces, including those of indwelling medical devices, and form biofilms made up of extracellular polymers. In this state, microorganisms are highly resistant to antimicrobial treatment and are tenaciously bound to the surface. To better understand and control biofilms on indwelling medical devices, researchers should develop reliable sampling and measurement techniques, investigate the role of biofilms in antimicrobial drug resistance, and establish the link between biofilm contamination and patient infection.

Current practice is to use a new sterile catheter with each insertion. A catheter to be re-used needs to be designed for biofilm removal and sterilization. Attempts to “engineer out the risks” have not been very successful. Coating the catheter has had some effect on infection rates.

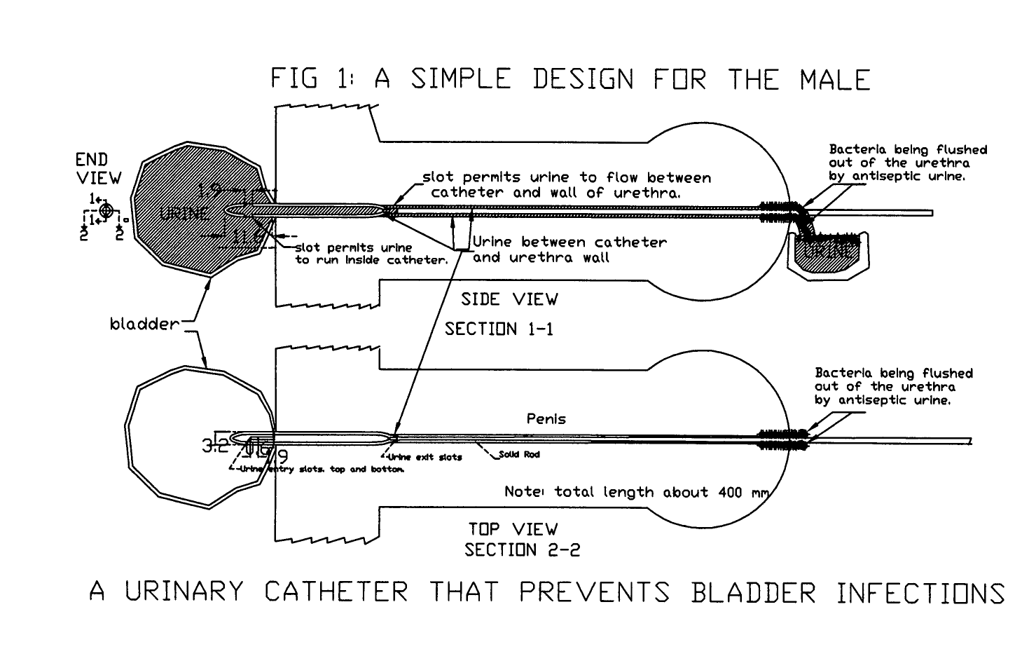

Of special interest was FIG. 1 that illustrates the paths the pathogens take to cause bladder infections. The authors did not consider the possibility that no “path” is necessary but that pathogens are already in place in the entrance to the urethra. In that case, the catheter prevents the pathogens from being washed out as would happen with normal urination. While deviations from acceptable standard practice, such as locating the urine bag higher than the patient's bladder, can cause infection, we may assume that such events are rare. If so, then most infections must be the result of the bacteria, usually present within the first half inch or so of the urethral orifice, making their way unimpeded from orifice to bladder. Bladder inflammations or Cystitis is generally attributed to pathogens entering the urethra from the exterior and traveling to the bladder. Also noteworthy is that nothing remotely resembling the devices describes in this patent application is mentioned under Novel Technology.

When a catheter is to be installed for any length of time, the designs herein proposed could be modified to be anchored in place to permit washing of the walls of the urethra. However, a continuous dribble may not be as effective in washing out bacteria as a periodic flush. When the risk of biofilm formation is considered, it may be that only intermittent catheterization use is feasible. When a new catheter is used for each urination then biofilms are not relevant, but any catheter that remains inplace is an infection risk. The longer the time, the greater is the risk. Some of the designs herein presented for the female and male, have no hollow spaces and could be scrubbed sterilized and re-used. This is not feasible for tubular catheters in current use.

### A BRIEF SUMMARY OF THE INVENTION

The object of the invention is to restore the natural washing out of bacteria in the urethra that occurs in normal urination. All currently available catheters are tubes prevent this natural defense from working. To allow the urine to wash away any pathogens lurking in the urethra it is necessary to cause or allow the urethra to be dilated while providing a path contiguous to the urethra walls so that the urine can wash all or some part of it.

### For the Male Only

The first and simplest design proposed takes advantage of the male anatomy. The head of about two inches in length dilates the urethral sphincter and has holes to allow urine to pass through the sphincter and into to the urethra within the penis. The head is then attached to a slim solid rod allowing the urine to pass between the slim rod and the walls of that portion of the urethra in the penis. Urination three or four times per day should be more than sufficient to keep bacteria from making the migration from urethral orifice to bladder, a distance of about six inches. As an option, a tailpiece to confine the urine back into a tube for easy disposal may be added and would be inserted about a half inch into the external urethral orifice.

### For the Female or the Male

A different design is necessary for the female. The female urethra is short and has a muscular sphincter. A design that dilates the sphincter and allows washing of the walls at the same time must take into account the propensity of the muscular walls to conform to the shape of the catheter. The catheter proposed for the female is a solid cylinder about two inches long with a streamlined point at one end and a handle at the other. The surface of the catheter can be any configuration that permits urine to wash the urethra walls. Varieties of possible configurations are presented in the section “Description of the Invention”.

The unique features of the invention are those features that cause the walls of the urethra to be washed by the urine being excreted during urination. For the female, the traditional tube is replaced by a solid object so made to permit the urine to flow out of the bladder and between the walls of the urethra and the catheter. For the male, a short flow-through head is attached to a thin rod that permits urine to flow in the space between the walls of the urethra and the thin rod. A preliminary patent search indicates that no catheter with these features exists.

A great variety of specific designs would allow the walls of the urethra to be washed during urination with a catheter. Any of these are “the invention.”

### DESCRIPTION OF THE INVENTION

### 1. A Simple Design for the Male

See FIG. 1: Design 1: A Simple Design for the Male.

The male urethral canal in the penis is ribbon shaped and of spongy tissue, not muscle. The catheter proposed has a hollow head section about 2 inches long, streamlined front and back for ease of entry and withdrawal. This head is attached to a thin shaft. The head has holes fore and aft to permit urine to flow through it. The pressure from the bladder is normally sufficient to dilate the urethra in the penis and allow urine to pass. This is what happens in normal urination. For this to occur using a catheter, the shaft must be small in diameter, (about 3 mm) so as not to dilate and stretch the urethra and prevent the urine from flowing. The shaft needs to be stiff enough to push the head through the sphincter, but soft and large enough to perform without any damage to the urethra. This design would allow most of the length of the urethra to be periodically washed down, preventing bacteria from entering the bladder. This design takes advantage of the male penis, which provides a generous length of urethra to be periodically washed. Urine pressure is sufficient to dilate the urethra and permit urine to pass. Periodic washing of this length of urethra should be enough to prevent any pathogen from traveling to the bladder.

Convenience Add-On For Design 1.

As a convenience, the shaft could be attached to a tubular tail section with holes to permit urine to flow into the tube, and an opening at the end for convenient urine disposal. This tail section would occupy the last inch or less of the penis and prevent urine from spraying about and confine it into a tube. It might be desirable to make the shaft inlayers with a stiff center and a soft slippery outer coating. The entire device could be coated with a soft coating. The coating could be impregnated with a slippery anti-bacterial substance to ease of entry and prevent accidental introduction of bacteria.

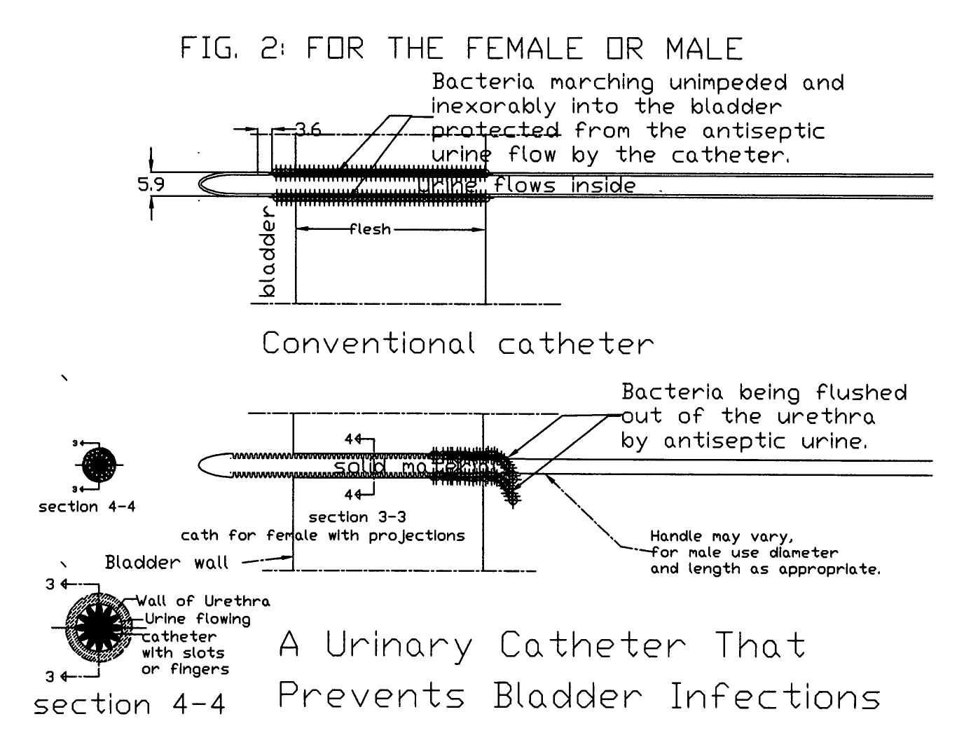

See FIG. 2: A Design for the Female

The female urethra consists of muscle as well as spongy tissue and a mucous coating. This sphincter muscle keeps the urethra closed. The inability to open the urethra is why the catheter is necessary. The device described above will not work for the female.

Smooth Grooves

The head consists of a solid cylindrical device, streamlined fore and aft, with slots that allow urine to wash at least some of the urethra walls without injuring delicate tissue. An optimal design would maximize urine flow rates and the ratio of washed to unwashed urethra wall area. A variety of geometry's could be sued t accomplish this. The device needs to be developed in detail to maximize comfort, and washing effectiveness, safety in use being an overriding condition. For the female, a convenient handle is attached. The head of the device might include some feature to contain or control the urine spray. For some, sitting on a toilet, bedpan or standing at a sink might be convenient.

Tall Pimples

3. Instead of slots, an array of fingers projecting radially outward to dilate the urethra walls while urine flushes through and between the fingers. An optimal geometry might have few or many fingers or projections. It might resemble a brush but with spherical tips to provide minimal but adequate pressure to dilate the urethra without injury or discomfort.

Screw

An Archimedes screw inside a flexible skin would provide a path for urine flow but pump when pressures are low, such as with an artificial bladder. If sufficiently smooth and slippery, the flexible sheath might not be necessary. The screw could be turned by hand or motor to move urine from bladder to outside, but at the same time, causing urine to wash the urethra wall.

Sponge

The device could be a solid shaft with an external coating of permeable, spongy material or made entirely of a spongy material that is strong and slippery enough to be inserted but permits urine to flow through it and wash the urethra walls. The device might be made in layers with the more permeable layer outside.

Cage

The head can be a cage exposing most of the wall to the urine flow, but smooth entry and withdrawal without snagging might be a problem.

Pop-outs

A hollow cylindrical head, small in diameter, for ease of entry and withdrawal, is attached via tubing to a syringe containing water. After insertion, the syringe is depressed causing an array of projections to expand radially outward to dilate the bladder by the amount necessary (within limits) to obtain a satisfactory urine flow.

Dynamic Devices, Wave Machines

The pop-out device can have its pop-ups arrayed in a pattern and connected with small tubing to separate pressure sources so that the popping-out and popping-in can be controlled by and external programmable source. It then becomes possible to create longitudinal wave motion. A peristaltic motion device that permits or pumps urine from the bladder might be useful, especially if the subject has an artificial bladder.

The CDC article referenced above estimates the cost of a bladder infection at $1000, an expensive consequence of using an inappropriate $1 catheter. Aside from the misery and death, the high costs justify liberal expenditures for prevention, maybe even a urethra washing machine.

1. A urinary catheter constructed so that surface areas of the urethra are washed with urine.
